(19) United States
(12) Patent Application Publication
(10) Pub. No.: US 2024/0374124 A1
(43) Pub. Date: Nov. 14, 2024

(54) HANDPIECE FOR A FLEXIBLE ENDOSCOPE OR FOR A FLEXIBLE ENDOSCOPIC INTRUMENT

(71) Applicant: KARL STORZ SE & CO. KG, Tuttlingen (DE)

(72) Inventors: Alexander FUCHS, Steißlingen (DE); Tobias LOTTES, Helmbrechts (DE)

(73) Assignee: KARL STORZ SE & CO. KG, Tuttlingen (DE)

(21) Appl. No.: 18/554,918

(22) PCT Filed: Apr. 11, 2022
(86) PCT No.: PCT/EP2022/059652

§ 371 Date: Oct. 11, 2023

(30) Foreign Application Priority Data
Apr. 12, 2021 (DE) 10 2021 109 025.3

(51) Int. Cl. A61B 1/005; A61B 1/00

(52) U.S. Cl. CPC A61B 1/0052; A61B 1/0011; A61B 1/0057

(57) ABSTRACT

A handpiece according to the invention for a flexible endoscope or a flexible endoscopic instrument which comprises a shaft with a shaft portion that can be set at an angle by means of at least one Bowden cable has a control element for actuating a pull element of the at least one Bowden cable, wherein the handpiece comprises at least one clamping plate which has at least one clamping slit and/or at least one clamping groove for clamping a Bowden cable sheath of the at least one Bowden cable. The invention also relates to a flexible endoscope and a flexible endoscopic instrument and also to a method for assembling a flexible endoscope or a flexible endoscopic instrument.

### CROSS-REFERENCE TO RELATED APPLICATIONS

This application is the U.S. national stage of PCT/EP2022/059652 filed on Apr. 11, 2022, which claims priority of German Patent Application No. 10 2021 109 025.3 filed on Apr. 12, 2021, the contents of which are incorporated herein.

### TECHNICAL FIELD

The present disclosure relates to a handpiece for a flexible endoscope or for a flexible endoscopic instrument according to the preamble of claim 1, to a flexible endoscope and a flexible endoscopic instrument, and also to a method for assembling a flexible endoscope or a flexible endoscopic instrument.

### BACKGROUND

Flexible endoscopes have a flexible, elongate shaft that is suitable for insertion into a cavity, such as a cavity inside the body or a cavity of a technical object. In the region of the distal end of the endoscope shaft (i.e. the end remote from the user), imaging optics are arranged in order to generate an image of a scene within the cavity, in which case, according to a common design, an electronic image recorder is arranged in the distal end region for the purpose of recording the image, the signals of which image recorder are transmitted, via electrical lines running within the shaft, to a handpiece arranged at the proximal end region of the shaft (i.e. the end region near the user). Such a flexible endoscope can also contain an illumination system for illuminating the cavity to be observed and, if necessary, one or more working channels for carrying endoscopic working instruments from the proximal to the distal end region of the shaft, and also suction and irrigation channels.

Endoscopic instruments are also known which comprise a flexible, elongate shaft that is also suitable for insertion into such a cavity. Such a flexible endoscopic instrument can be used to carry out manipulations in the cavity, for which purpose a tool can be arranged at the distal end of the shaft, which tool can be operated by means of an operating element via a transmission means running within the shaft, which operating element is located on a handpiece arranged at the proximal end of the shaft. Such a flexible endoscopic instrument does not generally have its own optics for recording an endoscopic image, but can in particular be used together with a flexible endoscope.

Flexible endoscopes and flexible endoscopic instruments are typically used for observing or performing manipulations in a cavity that can be reached via a curved access path or, for example, to observe the interior of an organ that itself has a curved shape. By virtue of its flexibility, the shaft can adapt to the curvature of the access route or of the organ. However, it is often desirable to be able to actively bend the distal end of the shaft, i.e. the tip of the endoscope or of the endoscopic instrument, in order to facilitate the introduction of the endoscope or of the endoscopic instrument through a channel that does not run in a straight line, in order to be able to move the tip within a cavity in a lateral direction, and to be able to change the viewing direction of the imaging optics or the orientation of a tool. For this purpose, the shaft has a controllable portion, in particular a controllable end portion, which can be actively angled by a desired amount in a desired direction and can be controlled for this purpose using an actuating element arranged on the handpiece.

To permit controllable bending of a shaft of a flexible endoscope or of a flexible endoscopic instrument, it is known to design it with a flexible basic structure, which can consist, for example, of links or segments which are arranged one behind the other and pivotably connected to one another by joints and which can be actuated via pull cables or Bowden cables guided in the endoscope shaft. The bending of the controllable portion from a straight shape to a curved or angled shape, and vice versa, can be controlled by axial movement of pairs of pull wires of the Bowden cables acting against each other. To actuate the pull wires, one or more handwheels or levers for example can be provided, which are arranged on the handpiece of the endoscope or of the endoscopic instrument. To be able to control the angle of the controllable shaft portion, the Bowden cable sheaths, in which the pull wires are guided in an axially displaceable manner, are fixed in the handpiece.

It is known for this that the Bowden cable sheath rests bluntly against a fixed stop in the handpiece. This has the disadvantage that any length tolerances of the Bowden cable sheath cannot be accommodated. Since Bowden cable sheaths with slightly different lengths have to be accommodated in the endoscope, undesirable bending of the Bowden cable sheath can occur, which can increase the friction between the Bowden cable sheath and the pull wire guided in the latter. Furthermore, with a blunt stop, the contact surface of the end face is often so small that the Bowden cable sheath, which is usually reinforced by steel, can press into a counter-support made of plastic, for example, which changes the pretensioning of the Bowden cable. This has to be prevented by using a correspondingly hard counter-support, which entails disadvantages in terms of costs and assembly effort.

US 2020/0229684 A1 discloses a handpiece for an endoscope, the Bowden cable sheaths being glued with adhesive into through-holes of an anchor block which is arranged within the handpiece. However, because of the necessary hardening of the glue, such adhesive bonding processes result in an increased amount of time during assembly. In addition, if the crosslinking is incomplete, the adhesives can subsequently release undesirable substances and, if the Bowden cable sheath is in an unfavorable position, it cannot be ruled out that the pull wire will be glued to the sheath.

Furthermore, the known handpieces have the disadvantage that only Bowden cables with a single, predetermined external diameter of the Bowden cable sheath can be fixed in them. On the other hand, endoscopes with different shaft diameters require the use of Bowden cables having a correspondingly adapted cross section. The fact that a correspondingly adapted handpiece has to be provided for each shaft diameter of the endoscope can result in considerable manufacturing and storage costs.

### SUMMARY

It is an object of the present disclosure to provide a handpiece for a flexible endoscope or for a flexible endoscopic instrument, whereby the disadvantages mentioned above are avoided as far as possible. In particular, it is an object of the disclosure to provide such a handpiece in which the connection to the at least one Bowden cable of the flexible endoscope or of the flexible endoscopic instrument is improved in such a way that, for example, a simple compensation of tolerances is made possible during assembly and/or adhesive bonding processes can largely be avoided, and/or assembly is made possible with lower costs and/or increased process reliability. Furthermore, it is an object of the present disclosure to provide a corresponding flexible endoscope and a flexible endoscopic instrument and also a method for assembling a flexible endoscope or a flexible endoscopic instrument.

This object is achieved by a handpiece according to claim 1, by a flexible endoscope or a flexible endoscopic instrument according to claim 16, and by a method according to claim 19.

Advantageous developments of the disclosure are set out in the dependent claims.

A handpiece according to the disclosure is designed as a handpiece for a flexible endoscope or a flexible endoscopic instrument, which endoscope or endoscopic instrument comprises a shaft with a shaft portion which can be angled by means of at least one Bowden cable and which is therefore also referred to as a controllable shaft portion: the shaft can comprise further shaft portions. The shaft is preferably elongate and designed for insertion into a cavity, for example into an internal cavity of a human or animal body or into a cavity of a technical or other object. The handpiece can be designed as a part or assembly of a flexible endoscope or of a flexible endoscopic instrument and can be assembled with the shaft and possibly together with other components to form such an endoscope or instrument, the handpiece being connected to a proximal end of the shaft. The handpiece can also be referred to as a handle or grip. The flexible endoscope or the flexible endoscopic instrument is designed in particular for use in medical, in particular surgical, procedures or for technical applications. Preferably, the handpiece is designed for single use, for example for a disposable endoscope or a disposable instrument.

The angle of the controllable shaft portion can be controlled by means of the at least one Bowden cable. The Bowden cable comprises a Bowden cable sheath and a pull element, such as a pull wire or a pull cable, which is guided therein in an axially displaceable manner at least in sections: where the term “pull wire” is used below; a pull cable can also be provided instead. In particular, the Bowden cable sheath extends as far as the proximal end of the controllable shaft portion and is fixed there, while the pull element guided in the Bowden cable sheath extends as far as the distal end of the controllable shaft portion and is fixed there. By axial displacement of the pull element relative to the Bowden cable sheath, the controllable shaft portion can be actively angled by a desired amount in a desired direction. Preferably, the controllable shaft portion can be angled by means of two or four pull wires acting in pairs against one another, each of which is guided in the manner of a Bowden cable in a Bowden cable sheath running through the first shaft portion and is fixed in the distal end region of the shaft.

The handpiece according to the disclosure has a control element for actuating the pull element of the at least one Bowden cable, in particular for moving or pulling the pull wire of the at least one Bowden cable in the axial direction relative to the Bowden cable sheath. The control element can be, for example, a control wheel over which a proximal end portion of the pull element can be guided or held thereon and with which the pull element can be adjusted in the axial direction. If two pull elements acting against each other are provided, they can be guided on opposite sides onto the control wheel, whereby they can also be connected to each other on the proximal side and can form a continuous pull element. Furthermore, the handpiece can comprise an actuating element, for example a handwheel or a lever, with which the control element can be actuated to move or pull the pull element, for example the control wheel can be rotated. If the handpiece is connected to the shaft of the flexible endoscope or of the flexible endoscopic instrument, the pull element of the at least one Bowden cable can be axially displaced through actuation of the control element by means of the actuating element, and the controllable shaft portion can thereby be angled. In the following, “axial” refers to an axis of the respective Bowden cable, i.e. to a central axis of the Bowden cable sheath, in a relevant portion, while the term “longitudinal axis” refers to a longitudinal axis of the endoscope shaft or to a longitudinal direction of the handpiece, which can correspond to the axial direction of the at least one Bowden cable sheath upon entry into the handpiece, in particular the longitudinal direction of the shaft when the latter is connected to the handpiece in an elongate arrangement.

The handpiece can have further operating elements, for example an actuating element for a tool of an endoscopic instrument arranged at the distal end of the shaft and/or connections for other devices, for example a suction and irrigation device or a camera unit.

According to the disclosure, the handpiece comprises at least one clamping plate which has at least one clamping slot and/or at least one clamping groove for clamping the Bowden cable sheath of the at least one Bowden cable. The at least one clamping slot or the at least one clamping groove is in particular arranged and designed in such a way that a proximal portion of the Bowden cable sheath can be introduced into it, such that the Bowden cable sheath can be fixed therein by clamping. The proximal portion can be a proximal end portion of the Bowden cable sheath or a portion spaced apart from the proximal end of the Bowden cable sheath from which the pull wire guided therein emerges and continues to the control element. In particular, the handpiece is designed in such a way that the Bowden cable sheath can be fixed in the axial direction solely by being clamped in the at least one clamping slot or the at least one clamping groove. In particular, the at least one clamping slot can be open on one side at least in an assembled state of the handpiece. In a particularly advantageous manner, the at least one clamping slot or the at least one clamping groove can be designed such that the at least one Bowden cable sheath can be pressed into the clamping slot or the clamping groove transversely to the axial direction. For the sake of simplicity, often only the term “clamping slot” is used below, although a clamping groove can also be included.

By virtue of the fact that the handpiece comprises at least one clamping plate, which has at least one clamping slot and/or at least one clamping groove in which the Bowden cable sheath of the Bowden cable can be clamped, a connection of the Bowden cable sheath to the handpiece can be made possible in a simple manner so that, by moving the pull element relative to Bowden cable sheath, it is possible to control the angle of the controllable shaft portion. In particular, the Bowden cable sheath can be non-positively fixed in the handpiece by clamping it in the at least one clamping slot or the at least one clamping groove. It can be provided that the Bowden cable sheath can be fixed in a non-positive manner in the axial direction, in particular simply by clamping it in the clamping slot or in the clamping groove. In addition, in a direction transverse to the axial direction, the Bowden cable sheath can be held for example in a non-positive manner or in a non-positive and positive manner.

By virtue of the fact that the handpiece is designed for clamping the Bowden cable sheath in the at least one clamping slot or in the at least one clamping groove, a connection of the proximal end portion of the Bowden cable sheath to the handpiece can be made possible without an adhesive bonding process being needed for this purpose.

Furthermore, this makes it possible to compensate for any length tolerances of the Bowden cable sheath, with only an engagement on the outside of the Bowden cable sheath being necessary. In this way, undesirable bending of the Bowden cable, which would lead to increased friction of the pull element, can be avoided. Finally, a connection can be made by clamping using simple assembly processes, as a result of which the process reliability can be increased and the manufacturing effort can be reduced, which is particularly advantageous when assembling an endoscope or endoscopic instrument intended for one-time use.

When connecting the Bowden cable sheath to the handpiece by clamping it in the clamping slot or the clamping groove, it is possible to exploit the fact that the Bowden cable sheath, which is usually reinforced by steel, is generally elastically deformable, with relatively high stiffness, with respect to a cross-sectional deformation by a force acting transversely to an axial direction. It can thus be achieved that a sufficient force can be exerted on the Bowden cable sheath in the transverse direction in order to generate a high frictional force for non-positively fixing the Bowden cable sheath without impairing the displaceability of the pull element that is guided therein. The Bowden cable sheath is typically approximately cylindrical at least in sections and can, for example, have an external diameter in the range of less than 2 mm, in particular between approximately 1 mm and approximately 0.3 mm, for example approximately 0.75 mm or 0.43 mm.

According to a preferred embodiment of the disclosure, the clamping plate comprises at least one transverse wall, wherein the at least one transverse wall has the at least one clamping slot for clamping the Bowden cable sheath of the at least one Bowden cable. The clamping plate can, for example, comprise a base plate or bearing plate which supports the at least one transverse wall that has the clamping slot. The at least one transverse wall can be designed as a slat or rib that is arranged on the base plate. The at least one transverse wall can extend substantially transversely to an axial direction of the at least one Bowden cable or transversely to a longitudinal direction of the handpiece, parallel to which the at least one Bowden cable is guided from the shaft into the handpiece. The base plate can have a surface on which the at least one transverse wall is arranged, with the at least one Bowden cable sheath running substantially parallel to the surface of the base plate. The at least one transverse wall can be formed in one piece with the base plate: the clamping plate can be made in one piece overall and can be produced, for example, from plastic, for example as an injection molded part. The transverse wall can be interrupted and have several segments, wherein the at least one clamping slot can be formed between two adjacent segments, i.e. in this case the clamping slot is formed by an interruption or a gap in the at least one transverse wall. A clamping force exerted by the transverse wall can achieve a firm, non-positive fixation of the Bowden cable sheath.

Advantageously, the clamping plate can be designed for simultaneous clamping of at least two Bowden cable sheaths, the at least one transverse wall having at least two clamping slots for clamping the at least two Bowden cable sheaths in one clamping slot each. The at least two Bowden cable sheaths can, for example, be associated with two Bowden cables acting against each other, the pull elements of which can be adjusted to act against each other in order to angle the controllable shaft portion in the opposite direction. The two Bowden cables can be guided parallel to each other from the proximal end of the shaft into the handpiece, wherein the respective longitudinal axes of the Bowden cable sheaths define a common longitudinal plane, and wherein the at least one transverse wall is able to be perpendicular to the common longitudinal plane, and furthermore, if appropriate, a surface of the base plate that carries the at least one transverse wall can be directed parallel to the common longitudinal plane. This makes it possible to fix the Bowden cable sheaths of at least two mutually acting Bowden cables of the endoscope or of the endoscopic instrument in a particularly simple manner.

According to a particularly advantageous embodiment of the disclosure, the clamping plate has at least two transverse walls, each of which has at least one clamping slot for clamping the Bowden cable sheath of the at least one Bowden cable, wherein the clamping slots of the at least two transverse walls are arranged one after the other in an axial direction of the Bowden cable sheath of the at least one Bowden cable, preferably in a straight line, in particular pointing toward an attachment point of the pull element on the control element. The at least two transverse walls are arranged in particular parallel to each other, for example parallel to each other on the base plate of the clamping plate. In particular, the plurality of transverse walls running parallel to one another can be referred to as “slats”.

According to this embodiment, it has been recognized that, by clamping the at least one Bowden cable sheath in two clamping slots following each other in the axial direction, a high frictional force can be obtained for non-positively fixing the Bowden cable sheath, without the cross section of the Bowden cable sheath being deformed in a disadvantageous manner, and at the same time the non-positive fixing can be achieved in a simple manner by pressing the Bowden cable sheath into the at least two clamping slots. In a further advantageous manner, for example, three, four, five, six or more transverse walls can be provided, each with at least one clamping slot, whereby a further increase in the frictional force for fixing the at least one Bowden cable in the handpiece can be achieved without excessive deformation of the Bowden cable sheath.

In particular, the clamping plate can be designed to simultaneously fix two Bowden cable sheaths, which, as has been described above, can be used to angle the controllable shaft portion in opposite directions. If a plurality of transverse walls are provided, each of which has two clamping slots for clamping the two Bowden cable sheaths, the clamping slots of the plurality of transverse walls are each arranged one after the other in the axial direction of the Bowden cable sheath in question: if more than two transverse walls are provided, the respective clamping slots are preferably arranged following one another in a straight line. Since the pull wires of the two Bowden cables usually attach to the control element at a greater distance, for example guided tangentially onto a control wheel, than the distance between the Bowden cables when they enter the handpiece, the clamping plate is preferably designed to hold the two Bowden cable sheaths in an approximately V-shaped arrangement, with the two legs pointing in the direction of feed of the pull wires to the control element. In this case, the Bowden cable sheaths can run in a correspondingly curved shape on the distal side of the clamping plate in order to transition from the parallel arrangement into the V-shaped arrangement in the clamping plate when entering the handpiece.

According to one embodiment, it can be provided that the at least two transverse walls have at least one further interruption and are connected to one another in the axial direction of the Bowden cable sheath. The transverse walls can thus form strips with projections running along the Bowden cable sheath, between which the at least one Bowden cable sheath can be clamped, and which are therefore referred to here as clamping strips. If, for example, the clamping plate is designed to clamp two Bowden cable sheaths, the interrupted transverse walls connected to one another in the axial direction can thus form two pairs of paired parallel clamping strips, between the projections of which the two Bowden cable sheaths can be fixed by clamping. This allows a particularly rigid arrangement in order to achieve a particularly high clamping force.

Advantageously, the at least one clamping slot can be designed to taper toward a clamping region, starting from an opening of the clamping slot. This means that a width of the clamping slot decreases starting from the opening and is smaller in the clamping region than in a region adjacent to the opening. In particular, in the region of the opening, a bevel or a chamfer can be provided on one or both sides of the clamping slot or the clamping groove. As a result, assembly of the endoscope or of the endoscopic instrument can be further simplified, with the at least one Bowden cable being pressed into the clamping slot.

Advantageously, the clamping slot can be delimited on a proximal and/or distal side of the transverse wall by a bevel or a chamfer. The at least one clamping slot is formed in particular on one or both sides by wedge-shaped regions of the at least one transverse wall: these preferably each extend into an edge toward the clamping slot, wherein the Bowden cable sheath can be clamped between the edges formed on both sides. As a result, the frictional connection for fixing the Bowden cable sheath can be further improved, wherein the transverse wall does not have to run exactly perpendicular to the axial direction of the Bowden cable sheath.

The clamping plate can advantageously be designed for selectively clamping Bowden cable sheaths with different external diameters. A handpiece with a clamping plate designed in this way can thus be suitable for use with a first shaft which comprises a Bowden cable with a first external diameter of the Bowden cable sheath, and optionally instead with a second shaft which has a Bowden cable with a second external diameter of the Bowden cable sheath, wherein the second external diameter is different from the first external diameter: for example, the second external diameter can be greater than the first by 50%, 75% or 100% or more. Since the use of Bowden cables with a larger cross section is generally required for endoscopes with a larger shaft cross section, this means that the handpiece can be used for endoscopes with different shaft cross sections. The same applies to endoscopic instruments with different shafts. Such a modular structure enables a reduction in the variety of parts and the number of necessary production tools, which means that production and storage can be simplified and made more cost-effective.

According to one embodiment, the at least one clamping slot can be designed to be stepped and in particular can have at least two clamping regions with different widths. For example, a clamping region with a smaller width can be arranged closer to a base plate that supports the at least one transverse wall, and a clamping region with a larger width can be arranged closer to an opening of the clamping slot. This makes it possible in a particularly simple manner for the clamping plate to be designed for fixing Bowden cable sheaths of different diameters.

Alternatively or additionally, at least two clamping slots that have different widths can be provided for selectively clamping Bowden cable sheaths of different external diameters. In particular, it can be provided that one or each transverse wall has at least two clamping slots which have different widths in the respective clamping region: in the case where the clamping plate is designed to clamp two Bowden cable sheaths at the same time, at least two pairs of clamping slots can be provided, wherein the clamping slots of a pair are designed identically, but the different pairs have different widths. A handpiece equipped with such a clamping plate is also suitable for use with different shafts that have Bowden cables with different external diameters.

According to a particularly advantageous embodiment of the disclosure, the clamping plate has a plurality of transverse walls, each of which has at least a first pair and a second pair of clamping slots, wherein the clamping slots of the first pair have a first width and the clamping slots of the second pair have a second width different from the first. According to this embodiment, the clamping slots are arranged in the at least two transverse walls in such a way that, in a first installation position of the clamping plate, two Bowden cable sheaths held in the clamping slots with the first width point in a straight line on the proximal side of the clamping plate to the attachment points of the pull wires on the control element, so that these are, for example, guided tangentially to a control wheel, and, in a second installation position of the clamping plate, two Bowden cable sheaths held in the clamping slots with the second width are also directed in a straight line to corresponding attachment points on the control element. By changing between the installation positions, a first pair of Bowden cable sheaths with a first external diameter and alternatively a second pair of Bowden cable sheaths with a second external diameter can thus be fixed using the same clamping plate designed as a reversible part. In particular, the second installation position can be one in which the clamping plate is turned 180°, relative to the first installation position, about an axis perpendicular to the base plate. This makes it possible to achieve a modular structure in a particularly simple manner, whereby the handpiece can be adapted to different shafts by reversing the clamping plate.

According to an advantageous embodiment, the at least one clamping slot has a constriction which is arranged between an opening of the clamping slot and a clamping region and which has to be overcome when a Bowden cable sheath is pressed into the clamping slot. The constriction can be formed, for example, by latching projections arranged on both sides of the slot, which latching projections thus form an undercut that prevents the Bowden cable sheath from leaving the clamping slot. In addition to the friction on account of the clamping, the Bowden cable sheath can thereby be held particularly securely in the at least one clamping slot. The constriction is dimensioned in such a way that the Bowden cable sheath, when being pressed in, can overcome it without causing damage. If a plurality of transverse walls are provided, such a constriction can be provided in one or more successively arranged clamping slots, for example in the at least one clamping slot of the proximal transverse wall and the at least one clamping slot of the distal transverse wall, while the clamping slots in the remaining or intermediate transverse walls do not need to have such a constriction. Those clamping slots that are provided with latching projections do not necessarily also have to fix the Bowden cable sheaths in the axial direction.

Alternatively or additionally, it can be provided that the handpiece comprises at least one counter-holder, which closes the at least one clamping slot and thereby prevents a Bowden cable sheath clamped therein from being pushed out. After a Bowden cable sheath has been pressed in, the counter-holder can, for example, be placed onto an opening of the clamping slot or placed onto the clamping plate from the direction of the opening, in order to close the clamping slot. If several transverse walls are provided, the counter-holder can, for example, be designed to close the clamping slots of only one, several or all of the transverse walls. Several counter-holders can also be provided, which, for example, close the clamping slots of different transverse walls. Furthermore, the counter-holder can be adapted according to the external diameter of a pressed-in Bowden cable sheath or according to the press-in depth of the Bowden cable sheath. The counter-holder can be made of plastic and, for example, manufactured as an injection molded part. The at least one counter-holder can be a separate component or can also be formed by a component of the housing of the handpiece.

According to a preferred embodiment, the at least one counter-holder is designed as a further clamping plate, which can be designed as described above and which in particular comprises at least one transverse wall with at least one clamping slot. The at least one clamping slot of the counter-holder can be arranged and designed corresponding to the arrangement and design of the at least one clamping slot of the clamping plate. If the clamping plate has a plurality of transverse walls, each of which has at least one clamping slot, it can be provided in a particularly advantageous manner that the counter-holder is designed as a clamping plate with a plurality of transverse walls which engage between the transverse walls of the clamping plate when the counter-holder is placed onto the clamping plate, the clamping slots of the counter-holder being arranged corresponding to those of the clamping plate. By placing the further clamping plate for closing the openings of the clamping slots, it can thus be achieved that a Bowden cable sheath held in the at least one clamping slot is simultaneously clamped in the at least one clamping slot of the further clamping plate and can thereby be fixed in the axial direction with a particularly high force.

According to a further preferred embodiment, the clamping plate has at least one latching hook, which is designed to hold the at least one Bowden cable sheath in the at least one clamping slot. The latching hook can be elastically deformable in such a way that the latching hook deflects as the at least one Bowden cable sheath is pressed into the clamping slot and, when the Bowden cable sheath is pressed into the clamping slot or a clamping region of the clamping slot, springs back elastically and blocks the Bowden cable sheath in the clamping slot. The latching hook can be formed in one piece with the clamping plate.

This can also reliably prevent the Bowden cable sheath from slipping out of the clamping slot, with the further advantage that no additional component is needed for this.

Advantageously, it can additionally or alternatively be provided that at least one attachment point of the pull element on the control element is offset from a rectilinear continuation of an axial direction that the at least one Bowden cable sheath has in the region of the clamping plate, in such a way that, by pretensioning of the pull element, for example of the at least one pull wire, the Bowden cable sheath is preloaded into a clamping region of the at least one clamping slot or of the at least one clamping groove. In particular, the clamping plate can be designed for clamping two Bowden cable sheaths and the control element can be designed as a control wheel, with the pull wires of the two Bowden cables being guided on opposite sides into a circumferential groove of the control wheel. In this case, by means of an offset, which can also be achieved by tilting, of the groove of the control wheel relative to a plane in which the Bowden cable sheaths run in the clamping plate and which is for example parallel to a plane of a base plate of the clamping plate, it can be achieved that the Bowden cable sheaths are preloaded in the direction of the base plate by the pre-tensioning of the pull wires and are thus pulled into clamping slots which are formed in at least one transverse wall arranged on the base plate. This also allows the at least one Bowden cable sheath to be held in the at least one clamping slot, again with no additional component being needed.

The disclosure further relates to a handpiece for a flexible endoscope or a flexible endoscopic instrument which comprises a shaft with a controllable shaft portion which can be angled by means of at least one Bowden cable, wherein the handpiece has a control element for actuating a pull element of the at least one Bowden cable, and wherein the handpiece is designed for non-positively fixing the Bowden cable sheath of the at least one Bowden cable. The fact that the Bowden cable sheath can be fixed in a non-positive manner means in particular that the Bowden cable sheath can be fixed in the axial direction relative to the handpiece solely by force-fit or frictional connection. Preferably, the handpiece is designed for non-positively fixing the Bowden cable sheath by clamping a proximal portion of the Bowden cable sheath in at least one clamping slot or at least one clamping groove. In particular, the handpiece is designed as described above.

A flexible endoscope or flexible endoscopic instrument according to the disclosure comprises a shaft which comprises a controllable shaft portion that can be angled by means of at least one Bowden cable. The shaft is preferably elongate and designed for insertion into a cavity, for example into an internal cavity of a human or animal body or into a cavity of a technical or other object. The shaft can comprise one or more further shaft portions, in particular a first shaft portion, at the distal end of which the controllable shaft portion is arranged. The controllable shaft portion can therefore be angled, in particular actively curved, while the first shaft portion can be designed to be flexible, but not actively curveable. The flexible endoscope can have a camera head or the flexible endoscopic instrument can have an end effector, which can be arranged at the distal end of the controllable shaft portion. The flexible endoscope is in particular a medical endoscope, and the endoscopic instrument is in particular a surgical endoscopic instrument. The flexible endoscope or the flexible endoscopic instrument is preferably intended for one-time use, i.e. designed as a disposable endoscope or as a disposable instrument.

The at least one Bowden cable comprises a Bowden cable sheath and a pull element which is guided therein in an axially displaceable manner at least in sections. In particular, the Bowden cable sheath extends as far as the proximal end of the controllable shaft portion and thus, for example, through the first shaft portion, while the pull element guided into the Bowden cable sheath extends as far as the distal end of the controllable shaft portion. The distal end of the Bowden cable sheath can be fixed at the proximal end of the controllable shaft portion, while the pull element preferably extends beyond this and through the controllable shaft portion at least as far as its distal end region, where it is fixed.

Furthermore, the endoscope or the endoscopic instrument comprises a handpiece arranged at a proximal end of the shaft, which handpiece comprises a control element which is connected to the pull element of the at least one Bowden cable for actuating the at least one Bowden cable. The handpiece can be permanently or detachably connected to the proximal end of the shaft.

According to the disclosure, a Bowden cable sheath of the at least one Bowden cable is non-positively connected to the handpiece. In particular, the Bowden cable sheath is fixed on the proximal side in the handpiece by a non-positive connection, which engages on a lateral surface, in particular only on the lateral surface, of a proximal portion of the Bowden cable sheath. Advantageously, the Bowden cable sheath is fixed in the handpiece in the axial direction only by a non-positive connection. In particular, the handpiece has at least one clamping element for clamping the Bowden cable sheath, wherein the Bowden cable sheath is non-positively fixed by being clamped in or on the clamping element, for example by clamping two or more axially offset portions of the Bowden cable sheath. The at least one clamping element can be a separate component that is fastened in the housing of the handpiece, or it can be formed, for example, by a partial region of the housing.

The handpiece is preferably designed as described above, wherein the Bowden cable sheath is clamped in the at least one clamping slot or the at least one clamping groove of the clamping plate and is thereby non-positively connected to the handpiece. In particular, a clamping slot or a clamping groove of the clamping plate can, at least in one clamping region, have a width which is smaller than an external diameter of the Bowden cable sheath in a proximal portion of the Bowden cable sheath, wherein the width in the clamping region is matched to a material of the clamping plate, to an external diameter of the Bowden cable sheath and to a stiffness of the Bowden cable sheath related to a cross section, such that a clamping force acting on the Bowden cable sheath clamped in the clamping slot or the clamping groove is sufficient to fix it in the axial direction by friction, but at the same time the cross section of the Bowden cable sheath is only compressed so slightly that the pull element guided within the Bowden cable sheath can be moved within the Bowden cable sheath without increasing the friction.

According to a further aspect, the disclosure relates to a series of at least two flexible endoscopes, each of which is designed as described above, wherein the at least two endoscopes have different shafts with Bowden cables with different external diameters, but comprise similarly designed handpieces, each of which is designed to selectively clamp the Bowden cable sheaths with the different external diameters and in which the Bowden cable sheaths of the respective Bowden cables are fixed by clamping. The disclosure further relates to a series of at least two flexible endoscopic instruments, each of which is designed as described above and has different shafts with Bowden cables with different external diameters, but comprises similarly designed handpieces which are designed for selectively clamping the Bowden cable sheaths with the different external diameters and in in which the respective Bowden cable sheaths are fixed by clamping. The handpieces are designed, for example, for fixing Bowden cable sheaths with at least two different external diameters, by virtue of the fact that the at least one clamping slot or the at least one clamping groove is designed to be stepped, and/or the clamping plate has at least two clamping slots or clamping grooves of different widths. If the clamping plate has two pairs of clamping slots or clamping grooves with pairs of different widths, which can serve to clamp the Bowden cable sheaths of the corresponding width depending on the installation position of the clamping plate, the handpieces of the endoscopes or endoscopic instruments of the series can differ through the installation position of the clamping plate. Such a modular structure enables a reduction in the variety of parts and the number of necessary production tools, which means that production and storage can be simplified and made more cost-effective.

In a method according to the disclosure for assembling a flexible endoscope or a flexible endoscopic instrument, a shaft is provided for the endoscope or the endoscopic instrument, which shaft comprises a controllable shaft portion that can be angled by means of at least one Bowden cable, and a handpiece is provided that is designed as described above. The shaft can comprise the at least one Bowden cable, wherein a pull element and a Bowden cable sheath are or will be connected to a distal or a proximal end of the controllable shaft portion.

According to the disclosure, the Bowden cable sheath of the at least one Bowden cable is non-positively connected to the handpiece. The handpiece preferably comprises at least one clamping plate which has at least one clamping slot and/or at least one clamping groove for non-positively fixing the Bowden cable sheath of the at least one Bowden cable by clamping the Bowden cable sheath. In this case, the non-positive connection of the Bowden cable sheath to the handpiece is effected by pressing the Bowden cable sheath into the at least one clamping slot or the at least one clamping groove, in particular by pressing it in transversely to an axial direction of the Bowden cable sheath. To hold the Bowden cable sheath in the clamping slot or the clamping groove, latching projections can be arranged therein which the Bowden cable sheath overcomes when being pressed in and behind which it latches into place. This allows the Bowden cable sheath to be fixed in the handpiece in a simple and safe manner.

The method can comprise further steps: for example, the clamping plate can be fastened in the handpiece before or after being pressed in, and/or a counter-holder can be placed onto the clamping plate after the Bowden cable sheath has been pressed in. Furthermore, the pull element of the at least one Bowden cable can be connected to the control element of the handpiece, for example placed around a control wheel, so that the pull element can be actuated with the control element, i.e. can be displaced in the axial direction relative to the Bowden cable sheath. Furthermore, the handpiece can be connected to a proximal end of the shaft: in particular, a housing of the handpiece can be connected to the proximal end of the shaft via a coupling: alternatively, the shaft and the handpiece can be provided in an already connected form. The steps mentioned can also be carried out in a different order. For example, one or both of the latter steps can be carried out before or simultaneously with the non-positive connection of the Bowden cable sheath to the handpiece or the pressing into the clamping slot or the clamping groove.

Furthermore, the assembly of the Bowden cable in the shaft, in particular the distal connection of the pull element and of the Bowden cable sheath to the distal or proximal end of the controllable shaft portion, can be carried out completely or partially before or after the proximal connection of the pull element and/or of the Bowden cable sheath to the control element or to the handpiece. Furthermore, a housing of the handpiece can be closed and/or electrical lines or hose lines can be connected or attached.

Furthermore, it can be advantageously provided that, in order to additionally connect the Bowden cable sheath to the handpiece, the Bowden cable sheath and/or the clamping plate and/or an additionally introduced plastic material is at least partially heated. This means that the Bowden cable sheath can also be held in a form-fitting manner in a clamping slot or a clamping groove. The heating can take place in particular during or after the pressing in of the Bowden cable sheath. In this way, for example, it can be achieved that a transverse wall, which forms the clamping slot or the clamping groove and which can consist of a plastic material that is plastically deformable by heating, is heated and thereby deformed and partially flows around the Bowden cable sheath and thereby additionally holds the latter, in particular against being pushed out in a transverse direction from the clamping slot. It can be provided that a clamping slot or a clamping groove is only formed by pressing a heated Bowden cable sheath into the clamping plate. Alternatively or additionally, a plastic part can be pressed on and heated in the manner of hot caulking, such that the Bowden cable sheath is partially surrounded by the plastic material and is thus held in the clamping slot or in the clamping groove by an additional form-fit. This can also permit the additional advantage that the clamping plate is adapted to the cross section of the Bowden cable sheath during the pressing in of the Bowden cable sheath or during the hot caulking, such that the clamping plate can be suitable for different external diameters of the Bowden cable sheath.

It will be appreciated that the features mentioned above and the features yet to be explained below are applicable not only in the respectively specified combination but also in other combinations or on their own, without departing from the scope of the present disclosure.

Further aspects of the disclosure will become clear from the following description of preferred exemplary embodiments and from the attached drawing.

### BRIEF DESCRIPTION OF DRAWINGS

FIG. 1 shows a flexible endoscope with a handpiece according to one exemplary embodiment of the disclosure;

FIG. 2 shows the handpiece of the endoscope according to FIG. 1 in an opened view, with a clamping plate according to a first embodiment:

FIG. 3 shows the clamping plate of the handpiece according to FIG. 2 in a top view:

FIG. 4 shows a clamping plate of the handpiece according to a second embodiment in an oblique view:

FIG. 5 shows a clamping plate according to a third embodiment in an oblique view:

FIGS. 6a to 6c show a clamping slot or a transverse wall of a clamping plate in different representations: FIG. 7 shows a clamping plate according to a fourth embodiment in a top view:

FIGS. 8a and 8b show a clamping plate according to a fifth embodiment in two different installation positions:

FIG. 9 shows a clamping slot of a clamping plate according to a sixth embodiment in a view from the distal direction:

FIG. 10 shows a clamping plate with an attached counter-holder in a partially transparent view from the distal direction:

FIG. 11 shows an enlarged view of a clamping slot with an inserted Bowden cable sheath and an attached counter-holder:

FIG. 12 shows a clamping plate with an attached counter-holder according to a first variant of the counter-holder for two different Bowden cable sheaths in an oblique view:

FIG. 13 shows a clamping plate with an attached counter-holder according to a second variant of the counter-holder in a partially transparent oblique view:

FIGS. 14a to 14c show a clamping plate according to a seventh embodiment in three different representations:

FIG. 15 shows a variant of the seventh embodiment in schematic form:

FIG. 16 shows a clamping plate with an attached counter-holder according to a further embodiment of the disclosure;

FIGS. 17a and 17b show a clamping slot with an inserted Bowden cable sheath and a symbolically shown stamp for hot caulking with two different stamp geometries.

### DETAILED DESCRIPTION OF PREFERRED EMBODIMENTS

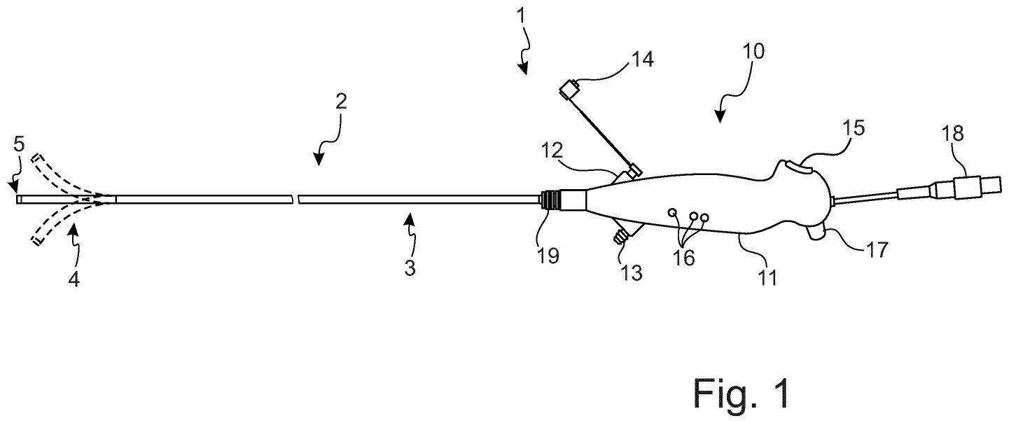

In FIG. 1, an embodiment of a flexible endoscope 1 according to the disclosure is shown in an overall view. The endoscope 1 has an elongate flexible shaft 2, which is designed for insertion into an internal cavity of a human or animal body. The shaft 2 comprises a first shaft portion 3, at the distal end of which, i.e. the end remote from the user, a second shaft portion 4 is arranged. The first shaft portion 3 is flexible and can, for example, adapt to the shape of a curved access path to an internal body cavity. As is indicated in FIG. 1, the second shaft portion 4 is controllable and can be curved from an elongate to an angled arrangement at least in one plane. A camera head 5 is arranged at the distal end of the controllable second shaft portion 4.

At a proximal end, the shaft 2 is connected to a handpiece 10, which serves to operate the endoscope 1 and for this purpose has an ergonomically shaped housing 11, on which operating elements and connections for further devices are arranged. In particular, the housing has an access port 12 for inserting a working instrument into a working channel that extends to the camera head 5, and also a hose connector 13 for connecting a suction hose, the access port 12 being closable with a closure cap 14 held on a flexible tab. Furthermore, a push button 15 for controlling the suction and a plurality of operating elements 16 for controlling other functions of the endoscope, such as light and zoom, are arranged on the housing 11.

Furthermore, the handpiece 10 comprises a control mechanism for bending the controllable second shaft portion 4, which control mechanism can be actuated by means of a lever 17 and is explained in more detail below. Furthermore, an electrical connection 18 is connected to the housing 11 via a cable, which is longer than depicted and which serves for connecting the endoscope 1 to a supply and evaluation device (not shown), in order to supply electrical and electronic components of the endoscope with electrical energy and to display and process a recorded endoscopic image. In the distal end region of the handpiece 10, in which the first shaft portion 3 is connected to the housing 11, a plurality of retaining lips 19 are arranged which securely hold the endoscope 1 during use in an insertion instrument.

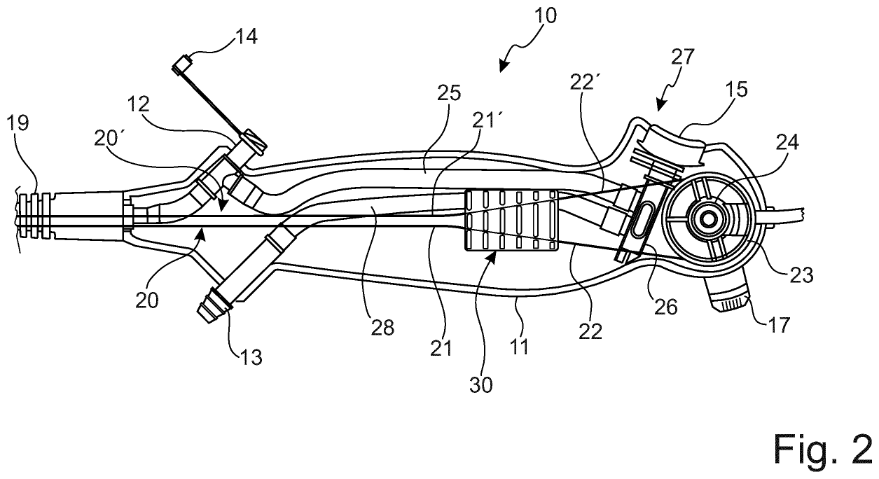

In order to be able to actively bend the controllable second shaft portion 4, the endoscope 1 comprises two Bowden cables 20, 20′, which are accommodated within the shaft 2 and which extend from the distal direction into the housing 11 of the handpiece 10 (see FIG. 2). The Bowden cables 20, 20′ each include a Bowden cable sheath 21, 21′, in which a pull wire 22, 22′ is guided in a longitudinally displaceable manner. The Bowden cable sheaths 21, 21′ run through the first shaft portion 3 to its distal end and are fixed there or at the proximal end of the second shaft portion 4. The pull wires 22, 22′ are guided further in the distal direction through the second shaft portion 4 and are fixed in the distal end region thereof or in the camera head 5. The pull wires 22, 22′ are arranged on opposite sides of the controllable second shaft portion 4, so that the latter can be angled in one direction or another by opposite axial displacement of the pull wires 22, 22′ relative to the respective Bowden cable sheath 21, 21′. The Bowden cable sheaths 21, 21′ and the pull wires 22, 22′ can be arranged and held at the distal end, for example as in the German patent application by the same applicant, filed on the same day as the present application and with the title “Shaft for a flexible endoscope or for a flexible endoscopic instrument” (internal file number: P20272DE), which in this regard is incorporated by reference into the present application.

As is indicated in FIG. 2, the Bowden cable sheaths 21, 21′ are fixed on the proximal side within the handpiece 10 by means of a clamping plate 30, which is supported on the housing 11 and connected thereto. At the proximal side of the clamping plate 30, the pull wires 22, 22′ are guided onto opposite sides of a control wheel 23, which is rotatably mounted in the proximal end region of the handpiece 10: the pull wires 22, 22′ can also be designed as sections of a continuous pull wire guided over the control wheel 23. The control wheel 23 is arranged in a rotationally fixed manner on a rotatable drive shaft 24, which is connected to the lever 17. By pivoting of the lever 17, the control wheel 23 can be rotated and the two counteracting pull wires 22, 22′ can be moved in opposite directions to each other. As a result, the bending of the controllable second shaft portion 4 can be controlled manually by a user of the endoscope 1.

The Bowden cable sheaths 21, 21′ are held in the clamping plate 30 in such a way that the pull wires 22, 22′ run in a straight line tangentially to a circumference of the control wheel 23: this ensures low-friction guidance of the pull wires 22, 22′ in the respective Bowden cable sheath 21, 21′. A proximal end of the Bowden cable sheaths 21, 21′, which is not shown in FIG. 2, lies in the region between the clamping plate 30 and the control wheel 23. Since the Bowden cable sheaths 21, 21′ are guided approximately parallel into the housing 11 of the handpiece 10 from the distal direction, they each run in a slight curvature on the distal side of the clamping plate 30, but this does not greatly increase the friction of the pull wires 22, 22′ in the Bowden cable sheath 21, 21′.

As can be seen in FIG. 2, further components are accommodated in the housing 11. In particular, the housing 11 accommodates a hose 25 which branches off from the access port 12 and through which the working channel running within the shaft 2 is connected to a first outlet of a valve housing 26 of the valve 27, which can be actuated with the push button 15. A second outlet of the valve housing 26 is connected to the hose connector 13 by means of a further hose 28, which is likewise routed within the housing 11.

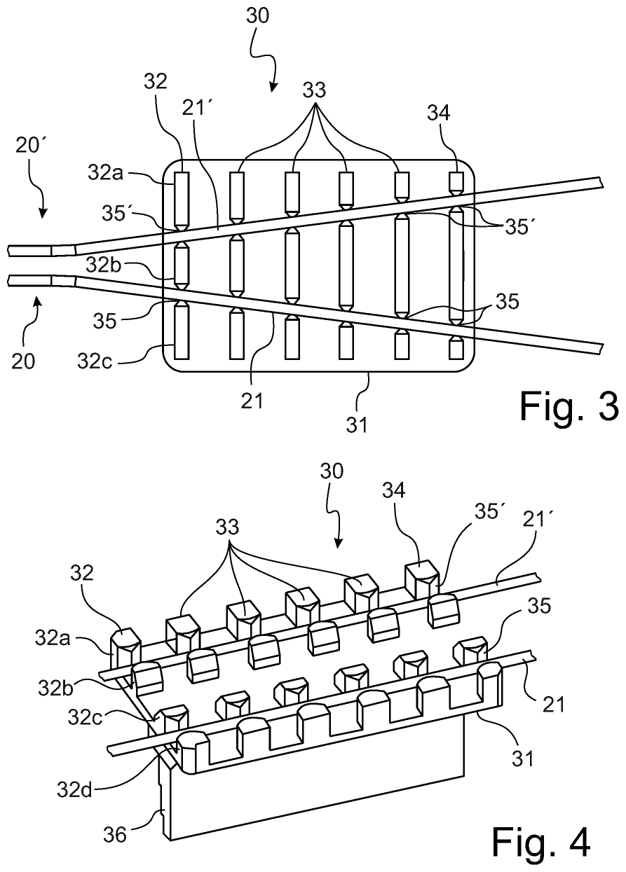

In FIG. 3, the clamping plate 30 of the handpiece 10, to which the Bowden cable sheaths 21, 21′ are non-positively fixed, is shown in an enlarged top view. The clamping plate 30 comprises a base plate 31, the surface of which visible in FIG. 3 lies parallel to a longitudinal plane spanned by the Bowden cable sheaths 21, 21′. Arranged on the base plate 31 are a plurality of mutually parallel slats which extend transversely to a longitudinal direction of the handpiece 10 and substantially transversely to an axial direction of the Bowden cable sheaths 21, 21′ and which are therefore referred to as transverse walls; in the example shown, the base plate 31 carries a distal transverse wall 32, four central transverse walls 33, and a proximal transverse wall 34. The transverse walls 32, 33, 34 can be formed in one piece with the base plate 31. The transverse walls 32, 33, 34 are each divided into segments, for example the distal transverse wall into segments 32a, 32b, 32c. Interruptions or slots are formed between the segments 32a, 32b, 32c and, in the exemplary embodiment shown, each extend to the base plate 31: alternatively, they could also end at a distance from the base plate 31. Each transverse wall 32, 33, 34 thus has two slots 35, 35′, with the mutually assigned slots 35 and 35′ of all transverse walls 32, 33, 34 each being arranged one after the other in a straight row: the two rows form an approximately V-shaped arrangement.

As is indicated in FIG. 3, the slots 35, 35′ are dimensioned such that the Bowden cable sheaths 21, 21′ can be pressed into them and held therein by clamping. The slots 35, 35′ are therefore also referred to here as clamping slots 35, 35′. The segments 32a, 32b, 32c are each designed tapering in a wedge shape toward the clamping slots 35, 35′, whereby a higher holding force can be achieved and, moreover, the Bowden cable sheaths 21, 21′ do not have to run exactly perpendicular to the transverse walls 32, 33, 34. By being accommodated in the slots 35, 35′ of the clamping plate 30, the Bowden cable sheaths 21, 21′ are held in an approximately V-shaped arrangement. This ensures that the pull wires 22, 22′, which are guided in the Bowden cable sheaths 21, 21′, run in a straight line to the control wheel 22, where they attach tangentially (see FIG. 2). The proximal end of the Bowden cable sheaths 21, 21′ is located between the proximal transverse wall 34 and the tangential attachment point of the pull wires 22, 22′ on the control wheel 23 and is not shown in FIG. 3 or in the other figures. It can also be seen in FIG. 3 that the Bowden cable sheaths 21, 21′, which are guided approximately parallel to one another from the handpiece 10 into the shaft 2 of the endoscope, run on the distal side of the distal transverse wall 32 in a slight curve to the clamping plate 30.

Since the Bowden cable sheaths 21, 21′ are pressed into the clamping slots 35, 35′ and are held therein in a frictional manner, the clamping plate 30 only engages non-positively on a cylindrical lateral surface in a proximal portion of the respective Bowden cable sheath 21, 21′. An exact axial position of the proximal end of the Bowden cable sheath 21, 21′ is therefore not specified, and it can therefore be located at almost any position between the proximal transverse wall 34 and the control wheel 23. During assembly, the Bowden cable sheaths 21, 21′ can therefore be pressed into the clamping slots 35, 35′ in such an axial position and thereby fixed, such that any length tolerances can be compensated, and unwanted curvatures or buckling of the Bowden cables 20, 20′ can be avoided.

In the example shown, the clamping plate 30 has a total of six slats or transverse walls 32, 33, 34. The frictional forces generated by clamping in the successive slots 35, 35′ of the transverse walls 32, 33, 34 interact and thereby generate a higher overall frictional force for fixing the Bowden cable sheaths 21, 21′. The force required to press the Bowden cable sheaths into each of the six clamping slots 35, 35′ is lower than it would have to be for a single clamping slot 35, 35′ in order to achieve the same frictional force. In addition, on account of the lower clamping force that acts in each of the slots 35, 35′ on the respective Bowden cable sheaths 21, 21′ in the transverse direction, excessive deformation of the Bowden cable sheaths 21, 21′ can be avoided and yet a high overall frictional force can still be provided. This can be used advantageously for the non-positive fixation of the Bowden cable sheaths 21, 21′ even if only two transverse walls 32, 34 were provided.

According to a second embodiment of the handpiece 10 according to the disclosure, the base plate 31 of the clamping plate 30, which is shown in FIG. 4, carries a total of six transverse walls 32, 33, 34, which however are each divided into four short segments or lamella stubs arranged in a transverse direction, for example the distal transverse wall 32 into the segments 32a, 32b, 32c, 32d. The clamping slots 35, 35′ are formed between in each case two pairs of segments 32a and 32b or 32c and 32d, in which the Bowden cable sheaths 21, 21′ of the Bowden cables 20, 20′ can be clamped. According to FIG. 4, the clamping slots 35, 35′ are arranged in rows running parallel to one another:

these can on the other hand also form a V-shaped arrangement corresponding to that shown in FIG. 3. FIG. 4 also shows a support 36 with which the clamping plate 30 is held on the housing 11 of the handpiece 10. The clamping plate 30 according to the second embodiment, and also the further embodiments described below, is installed in the handpiece 10 of the endoscope 1 instead of the clamping plate 30 according to the first embodiment, which endoscope I can otherwise be designed as described above.

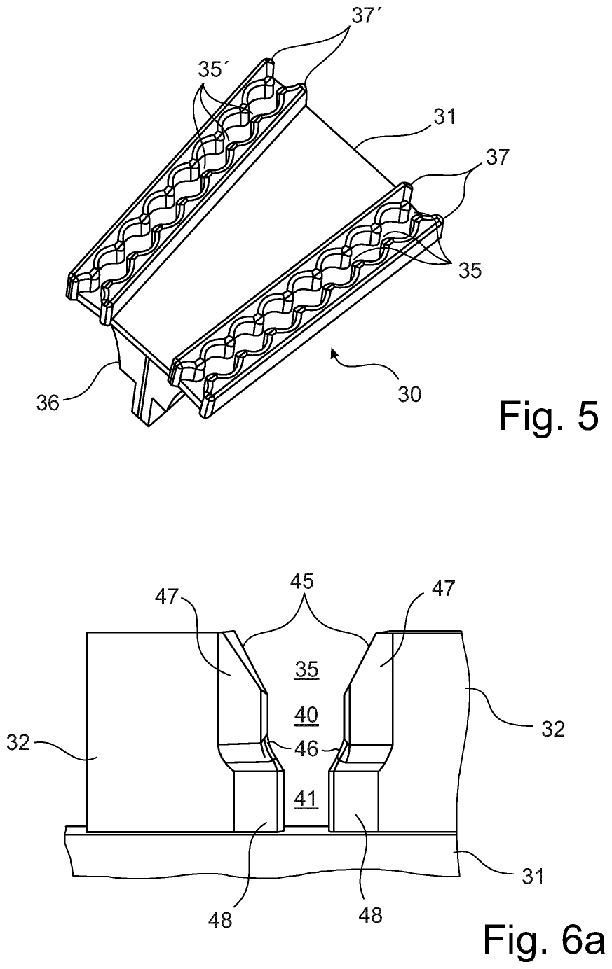

In the third embodiment of the clamping plate 30 shown in FIG. 5, the segments of the transverse walls 32, 33, 34, which are each arranged one after another in a straight row; are connected to form clamping strips 37, 37′, wherein the clamping slots 35, 35′, into which the Bowden cable sheaths 21, 21′ can be pressed, are formed in each case between two adjacent clamping strips 37, 37′ running parallel to each other. FIG. 5 shows an example of an arrangement with eight slots 35, 35′ each, in which the Bowden cable sheaths 21, 21′ can be fixed in a non-positive manner.

In the exemplary embodiments described above, the clamping slots 35, 35′ are each designed with a uniform width, such that Bowden cable sheaths 21, 21′ with an external diameter slightly greater than the width of the clamping slots 35, 35′ can be non-positively fixed in the clamping plate 30. As will be described below, the clamping plate 30 can also be designed for selectively fixing Bowden cable sheaths having several different external diameters.

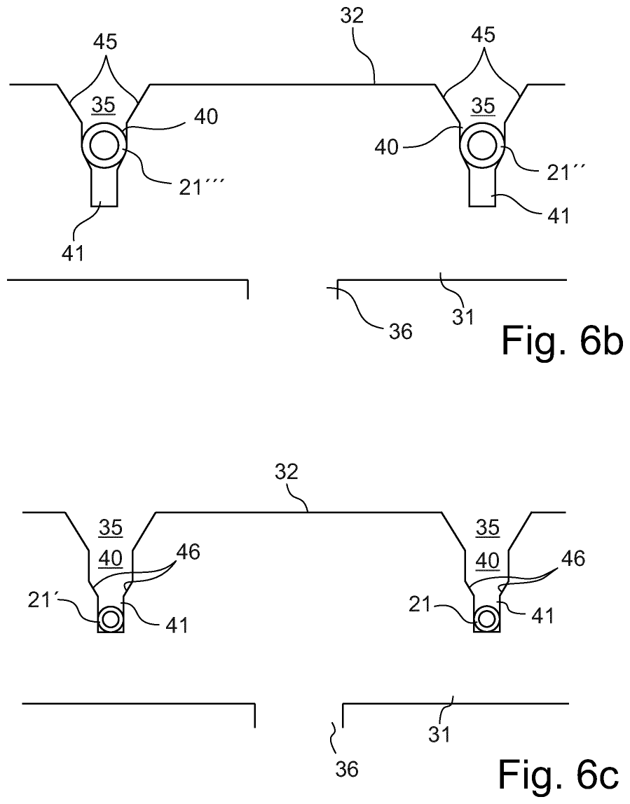

According to the exemplary embodiment shown in FIGS. 6a to 6c, the clamping slots 35, 35′ are stepped and each have a first and a second clamping region 40, 41. As is shown in FIG. 6a, the slot 35 has a greater width in the first clamping region 40 than in the second clamping region 41. The width of the first clamping region 40 is dimensioned such that a Bowden cable sheath 21″, 21″ with a greater external diameter can be pressed into the clamping region 40 and non-positively fixed therein, while the width of the second clamping region 41 is dimensioned such that a Bowden cable sheath 21, 21′ with a smaller external diameter can be pressed in and fixed therein in a non-positive manner (see FIGS. 6b and 6c). On account of the stepped design of the clamping slots 35, 35 ‘, the clamping plate 30 can thus be selectively used for the non-positive fixation of Bowden cable sheaths 21, 21’, 21″, 21″ of different diameters, which are pressed in to different depths, so that one handpiece 10 provided with the clamping plate 30 can be connected to different shafts which contain different sizes of Bowden cables. The first and second clamping regions 40, 41 can, for example, be suitable for clamping Bowden cable sheaths 21, 21′, 21″, 21″ with external diameters of approximately 0.75 mm and approximately 0.43 mm, respectively, and can accordingly be dimensioned slightly narrower, so that the transverse wall 32 generates a sufficient clamping force for non-positively fixing the Bowden cable sheath by deforming the sides of the clamping slot 35, 35′. This means that identical parts can be used for a series of endoscopes with different shaft lengths and diameters, which simplifies production and storage.

FIGS. 6a to 6c also show that, in order to facilitate the pressing in of the Bowden cable sheaths 21, 21′, 21″, 21″, the clamping slots 35, 35′ can be made wider in the region of the opening, i.e. in the upper region in the illustration according to FIGS. 6a to 6c, and in particular can have chamfers or insertion bevels 45 on both sides or a radius in each case. Even in the exemplary embodiments described above and in the following, the clamping slots 35, 35′ can each have such insertion bevels 45 in the region of their opening. In the step-shaped embodiment according to FIGS. 6a to 6c, insertion bevels 46 are also provided on both sides in the transition region between the first and second clamping regions 40, 41, in order to make it easier to press the Bowden cable sheath 21, 21′ into the second clamping region 41 of the clamping slot 35, 35′.

FIG. 6a also shows that the transverse wall 32 tapers in a wedge shape on both sides of the clamping slot 35 with wedge surfaces 47, 48, which are arranged in the region of the first and second clamping regions 40, 41 and can also extend into the region of the insertion bevels 45, 46. The wedge surfaces are arranged on both sides of the slot 35, as viewed in the axial or longitudinal direction. Furthermore, the wedge surfaces can be arranged on both the proximal and distal sides of the transverse wall 32 (see FIG. 3 for example). Even in the exemplary embodiments described above and below, the clamping slots can each be designed with corresponding wedge surfaces 47, 48. This means that the contact surface via which the clamping force acts on the Bowden cable sheath 21, 21′, 21″, 21′″ can be reduced, thereby making pressing in easier. Furthermore, this makes it possible to guide the Bowden cable sheaths 21, 21′, 21″, 21″ through the transverse walls 32, 33, 34 not exactly vertically, but at a corresponding angle for a tangential feed of the pull wires 22, 22′ to the control wheel 23, for example at an angle of approximately 10° with respect to a perpendicular to the transverse walls 32, 33, 34.

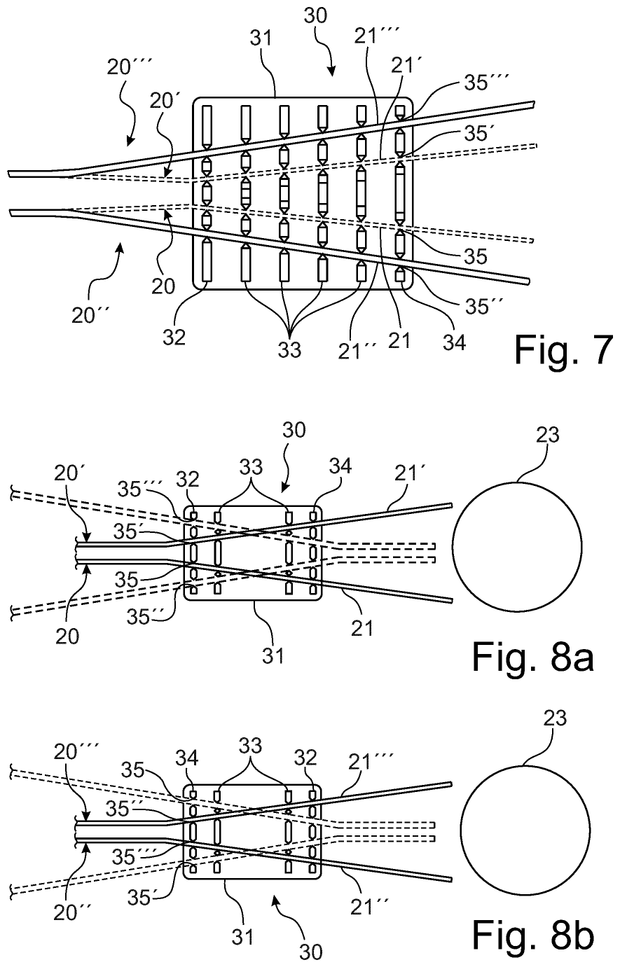

Two further embodiments of the clamping plate 30, which also enable selective use for fixing Bowden cable sheaths having different external diameters, are shown in FIG. 7 and FIGS. 8a and 8b, respectively. The transverse walls 32, 33, 34 are each divided into five segments, between which four clamping slots are formed in each transverse wall 32, 33, 34. Each transverse wall 32, 33, 34 has two slots 35, 35′ with a smaller width and two slots 35″, 35″ with a larger width. In the embodiment shown in FIG. 7, the clamping slots 35, 35′ with the smaller width are arranged one after the other in a V-shaped arrangement with a smaller distance between the legs, while the clamping slots 35″, 35″ with the larger width are arranged in a V-shaped arrangement with a greater distance between the legs. This allows either two Bowden cable sheaths 21, 21′ with a smaller external diameter to be fixed in the clamping slots 35, 35′ with the smaller width or two Bowden cable sheaths 21″, 21″ with a greater external diameter to be fixed in the clamping slots 35″, 35″ with the greater width.

In the embodiment of the clamping plate 30 shown in FIGS. 8a and 8b, the clamping slots 35, 35′, 35″, 35′ of the transverse walls 32, 33, 34 are arranged in oppositely open V-shaped arrangements. Depending on the direction in which the clamping plate 30 is installed in the housing 11 of the handpiece 10, two Bowden cable sheaths 21, 21′, 21″, 21″ having a smaller or greater external diameter can thus be fixed. In the installation position of the clamping plate 30 shown in FIG. 8a, the two Bowden cable sheaths 21, 21′ having a smaller external diameter are pressed into the clamping slots 35, 35′ and guided in a straight line tangentially to the control wheel 23; the Bowden cable sheaths 21″, 21″ having a greater external diameter, for which this installation position is not suitable, are only indicated by dashed lines. Alternatively, the clamping plate 30 can be fastened in the housing 11 in the installation position rotated through 180°, as is shown in FIG. 8b, in which installation position two Bowden cable sheaths 21″, 21′″ having a greater external diameter can be fixed non-positively in the clamping slots 35″, 35′″ and guided in a straight line tangentially to the control wheel 23: the Bowden cable sheaths 21, 21′ having a smaller external diameter, which could not be guided to the control wheel 23 in this installation position, are indicated by dashed lines in FIG. 8b. A handpiece 10 that has a clamping plate 30 designed in this way is therefore suitable for selectively connecting to different shafts which have differently dimensioned Bowden cables 20, 20′ or 20″, 20″, for which purpose the clamping plate simply has to be rotated through 180°.

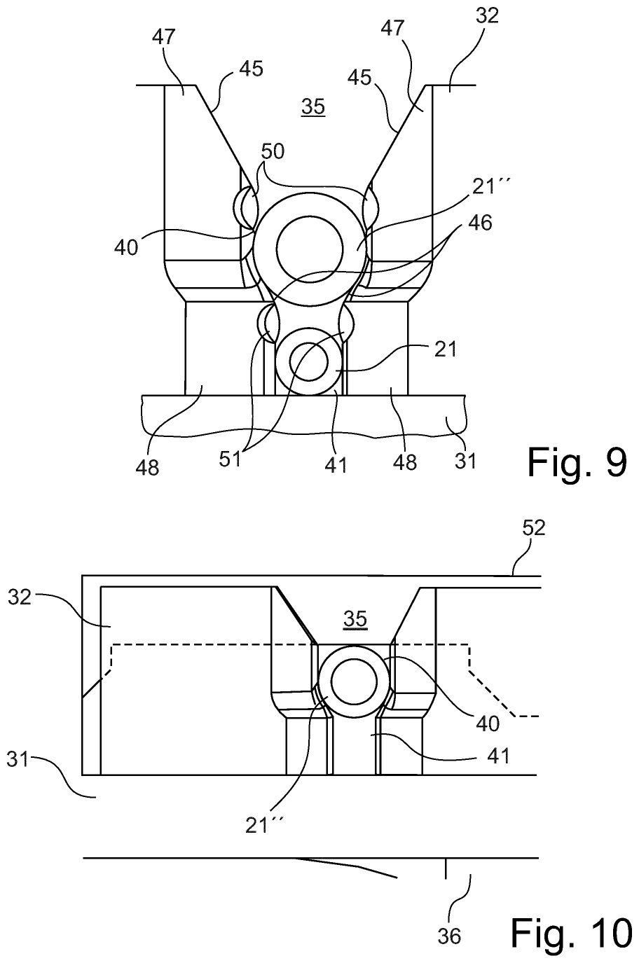

As is shown by way of example in FIG. 9, the clamping slot 35 can have one or more constrictions formed by latching projections 50, 51 which are attached to both sides of the slot 35 and which narrow the width of the latter. As is shown symbolically in FIG. 9, a Bowden cable sheath 21″ pressed into the clamping slot 35 is prevented by the latching projections 50 from slipping out of the clamping slot 35, this in addition to the clamping force itself. For this purpose, the latching projections 50 are arranged at the upper edge of the first clamping region 40. Alternatively, a Bowden cable sheath 21 having a smaller external diameter can be fixed in the second clamping region 41 in the clamping slot 35: it is additionally held in the clamping region 41 by the latching projections 51, which are arranged at the upper edge of the second clamping region 41. The respective constriction formed by the latching projections 50, 51 has to be overcome when the Bowden cable sheath 21, 21′ is pressed in, and it is therefore dimensioned such that an increased force is necessary to overcome the constriction, but such that the Bowden cable sheath 21, 21′ is not damaged when being pressed in.

In the previously and subsequently described embodiments of the clamping plate 30, the clamping slots can also be correspondingly equipped with latching projections 50, 51. It may be sufficient that, for example, only the clamping slots 35, 35′ of the distal transverse wall 32 and/or of the proximal transverse wall 34 are narrowed by such latching projections 50, 51, while the transverse walls 33 lying in between have no latching projections. For example, in the embodiment according to FIG. 4, the segments 32a, 32b, 32c, 32d of the distal transverse wall 32 and, in a corresponding manner, the segments of the proximal transverse wall 34 can each be designed with latching projections 50, 51 for holding the Bowden cable sheath 21, 21′ in the respective clamping slot 35, 35′. On the other hand, one or more of the transverse walls 33 located between the distal and proximal transverse walls 32, 34 can also have clamping slots 35, 35′ and corresponding latching projections 50, 51.

Alternatively or in addition to the latching projections 50, 51, a counter-holder can be provided, which also prevents the Bowden cable sheath from slipping out of the clamping slot. This is shown schematically in FIGS. 10 to 13. The counter-holder 52 is placed onto the base plate 31 from the direction of the opening of the clamping slot 35, i.e. from above in the schematic representation of FIG. 10, and blocks the Bowden cable sheath 21″, such that the latter cannot be readily pushed out of the clamping slot 35 or out of the first clamping region 40.

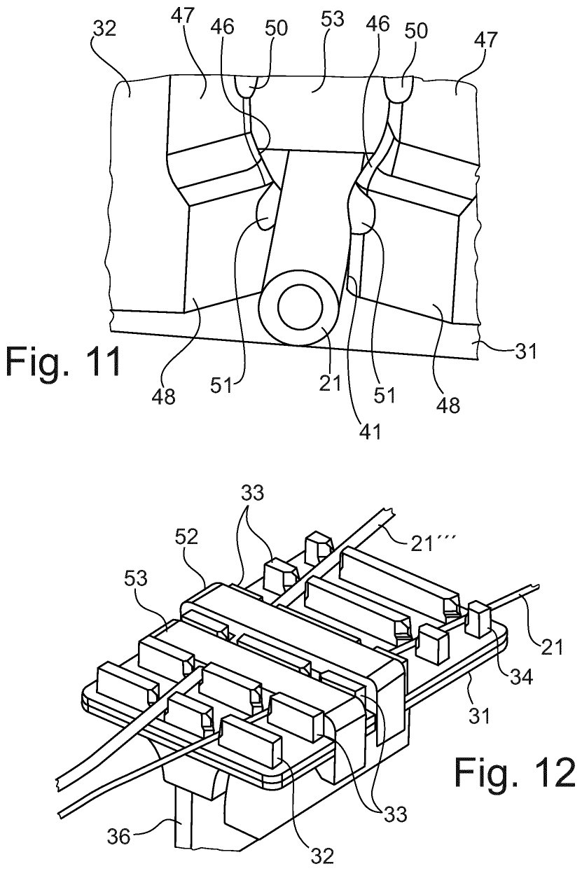

As is shown in FIG. 11, this safeguard can be provided in addition to the latching projections 50, 51 described above. FIG. 11 moreover shows that the counter-holder 53 is designed to secure a Bowden cable sheath 21 that is pressed into the second clamping region 41.

The counter-holder 52, 53 can, for example, be inserted between the transverse walls 32, 33, 34, as is shown in FIG. 12. FIG. 12 indicates that a counter-holder 52 of a first embodiment can be provided for securing a Bowden cable sheath 21″ which has a greater external diameter and is clamped in the first clamping region 40 (see FIG. 10), while alternatively a counter-holder 53 of a second embodiment can serve for securing a Bowden cable sheath 21 which has a smaller external diameter and is clamped in the second clamping region 41 (see FIG. 11).

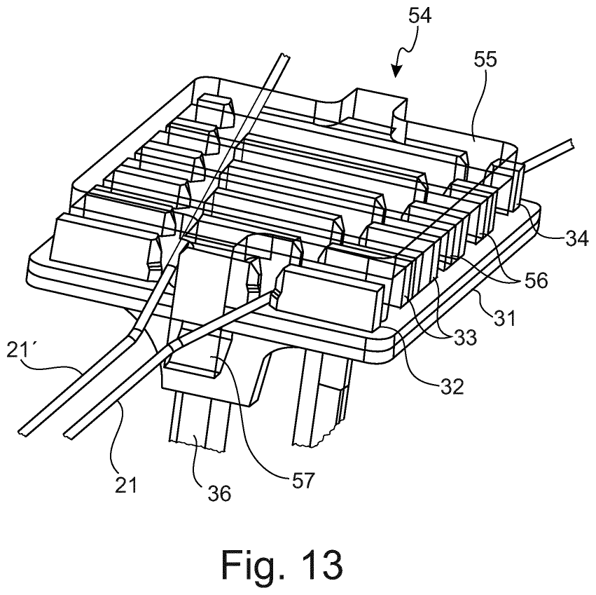

According to the exemplary embodiment shown in FIG. 13, the handpiece 10 comprises a counter-holder 54, which is designed in a similar manner to the clamping plate 30 with a base plate 55, on which a plurality of transverse walls 56 are arranged that have interruptions designed as clamping slots. By placing the counter-holder 54 onto the clamping plate 30 such that the transverse walls 55 of the counter-holder 54 and the transverse walls 32, 33, 34 of the clamping plate 30 correspondingly mesh, it is possible not only to obtain safety against pushing out in the transverse direction, but also additional clamping of the Bowden cable sheaths 21, 21′ and thus an additional increase in the frictional force for non-positively fixing the Bowden cable sheaths 21, 21′. As is also indicated in FIG. 13, the counter-holder 54 can be connected to the clamping plate 30 with the aid of latching hooks 57.

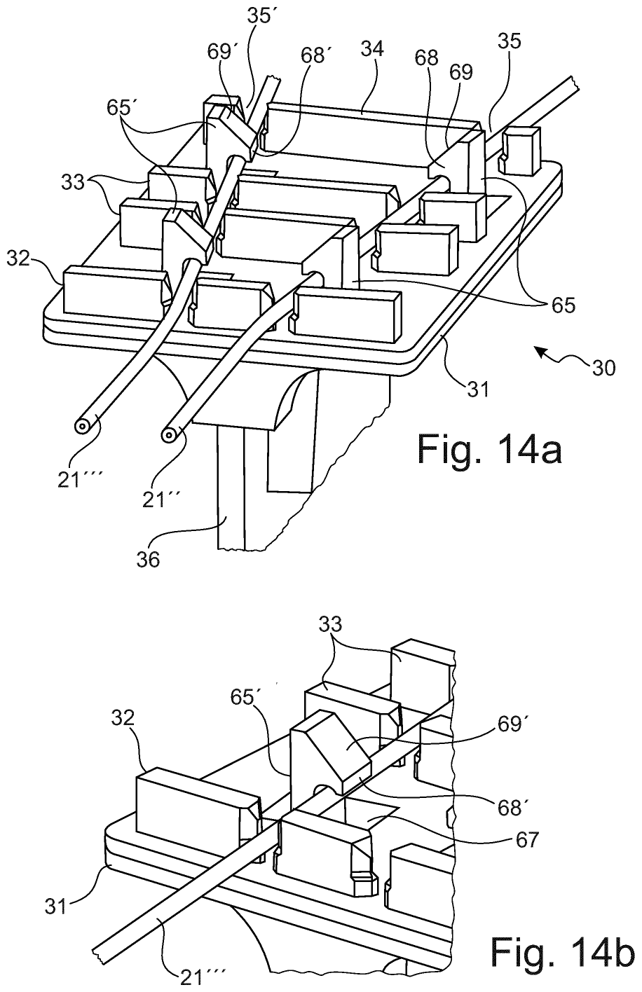

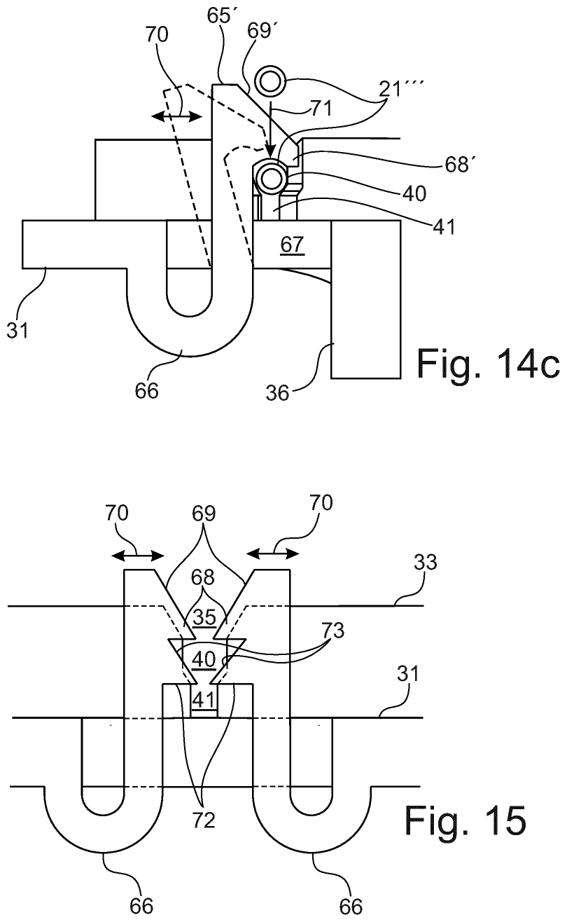

In the embodiment of the clamping plate 31 shown in FIGS. 14a to 14c, the Bowden cable sheaths 21″, 21″ are held in the clamping slots 35, 35′ by latching hooks 65, 65′, this in addition to the acting clamping force. As can be seen in the enlarged view shown in FIG. 14b and in the sectional view according to FIG. 14c, the latching hooks 65, 65′ are each attached to the base plate 31 from below via an arch 66 and engage through an approximately rectangular opening 67 of the base plate 31. At the upper end, the latching hooks 65, 65′ each have a latching lug 68, 68′, which engages over a Bowden cable sheath 21″, 21″ pressed into the slot 35, 35′ and thereby holds it in the clamping region 40. On its upper side, the latching lug 68, 68′ in each case has a run-on bevel 69, 69′.

As is indicated by the double arrow 70 in FIG. 14c, the latching hooks 65, 65′ are each arranged flexibly on the base plate 31 via the arch 66 and, in relation to the longitudinal direction of the handpiece 10 or an axial direction of the Bowden cable sheath 21″ “, can be deflected in a transverse direction, which is a horizontal direction in FIG. 14c. If the Bowden cable sheath 21” is inserted vertically from above into the clamping slot 35′ (arrow 71), a transverse force acts on the latching hook 65′ via the run-on bevel 69′. This therefore deflects in the transverse direction, such that the Bowden cable sheath 21″ can be inserted into the clamping slot 35′. When the Bowden cable sheath 21″ has reached the clamping region 40, the latching lug 68′ latches elastically behind the Bowden cable sheath 21″ and thereby holds the latter in the clamping region 40. The latching hooks 65 on the clamping slots 35 can be arranged in the transverse direction, acting counter to the latching hooks 65′ on the clamping slots 35′ (see FIG. 14a).

FIGS. 14a to 14c show a clamping plate 30 in which the clamping slots 35, 35′ are stepped in order to enable the fixation of Bowden cable sheaths 21, 21′, 21″, 21″ having two different external diameters, for example as shown in FIGS. 6a to 6c. In the example shown, the latching hooks 65, 65′ serve to secure the Bowden cable sheath 21′″, having the greater external diameter, in the first clamping region 40. Alternatively, the latching hooks 65, 65′ of a Bowden cable sheath 21′ can be designed with the smaller external diameter in the second clamping region 41 (not shown). According to a further variant (not shown), the clamping slots 35, 35′ can be designed to fix Bowden cable sheaths having only a predetermined external diameter, which sheaths can be held in the clamping slots 35, 35′ by appropriately designed latching hooks 65, 65′.

On the other hand, the latching hooks 65, 65′ can also be designed in such a way that, with a stepped design of the clamping slots 35, 35′, a Bowden cable sheath 21, 21′, 21″, 21″ having a greater or a smaller external diameter can be held in a respective clamping region 40, 41. Such an embodiment is shown schematically in a sectional view in FIG. 15.

According to FIG. 15, the latching hook 65 has two latching lugs 68, 72 arranged one above the other, which are arranged in such a way that the upper latching lug 68 can hold a Bowden cable sheath 21″ with a greater external diameter in the first clamping region 40 and, optionally, the lower latching lug 72 can hold a Bowden cable sheath 21 with a smaller external diameter in the second clamping region 41 (see also FIGS. 6a to 6c). The latching lugs 68, 72 each have a run-on bevel 69, 73 at the top side. When the Bowden cable sheath 21, 21″ is inserted into the respective clamping region 40, 41, the latching hook 65 is deflected over the run-on bevels 69 and 73 and springs back elastically after being pressed into the clamping region 40, 41 (double arrows 70). As a result, the Bowden cable sheath 21″ having the greater external diameter is blocked in the first clamping region 40 or the Bowden cable sheath 21 having the smaller external diameter is blocked in the second clamping region 41. FIG. 15 also shows that two latching hooks can be arranged opposite each other in order to hold the Bowden cable sheath 21, 21″ in the clamping slot 35. Otherwise, the clamping plate 30 can be designed as in FIGS. 14a to 14c.

The embodiments according to FIGS. 14a to 14c and FIG. 15 have the additional advantage that no counter-holder and therefore no further component is required in order to hold the Bowden cable sheath 21, 21′, 21″, 21″ in the clamping slot 35, 35′. In particular, the latching hooks 65, 65′ can be formed in one piece with the base plate 31.

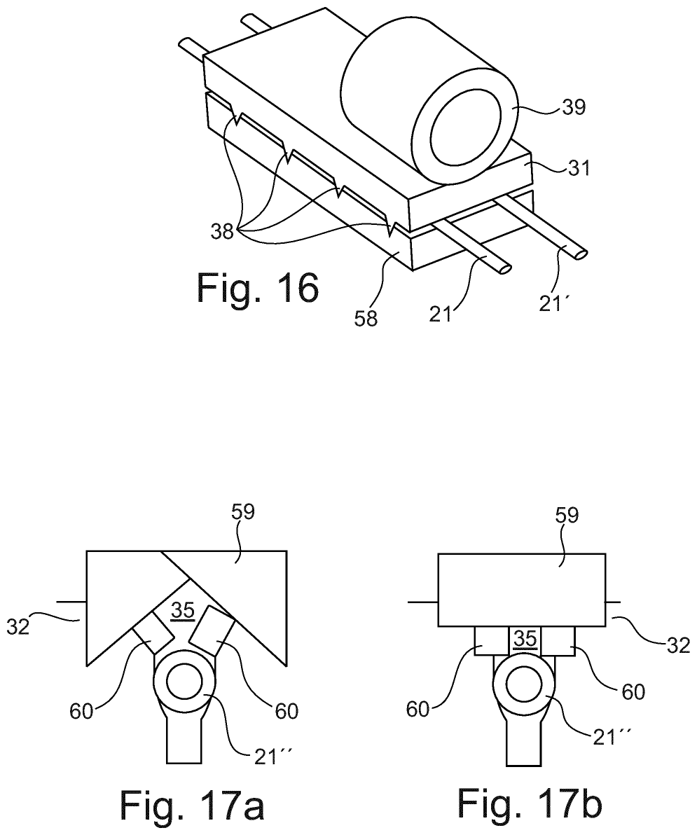

Furthermore, by heating the Bowden cables when they are pressed into the clamping slots, an additional safeguard against their being pushed out can be achieved by material of the clamping plate 30 or of the transverse wall melting and flowing around the Bowden cable sheaths. As is shown by way of example in FIG. 16, it can also be achieved that a guide of the respective Bowden cable sheath 21, 21′ is melted into the base plate 31 and/or into the counter-holder 58, and the clamping slots are also melted into the transverse walls; furthermore, it can be advantageous for the transverse walls to be designed as ribs 38 with a triangular cross section. As is also indicated in FIG. 16, an elastic support 39 of the base plate 31 can be provided in order to hold the latter and the counter-holder 58 against each other.

As is shown in FIGS. 17a and 17b, an additional safeguard against a pressed-in Bowden cable sheath 21″ being pushed out of the clamping slot 35 can also be achieved by hot caulking. In this case, by means of a heated stamp 59, plastic parts 60 are pressed for example obliquely (FIG. 17a) or vertically (FIG. 17b) from above against the pressed-in Bowden cable sheath 21″ and thereby melted, such that the plastic partially flows around the Bowden cable sheath 21″ and thereby additionally holds the latter in a form-fitting manner in the clamping slot 35.

For the sake of clarity, not all reference signs are shown in all of the figures. Reference signs not explained in the context of one figure have the same meaning as in the other figures.

1. A handpiece, for a flexible endoscope or a flexible endoscopic instrument which includes a shaft with a shaft portion configured to be angled by means of at least one Bowden cable, the handpiece comprising:
a control element for actuating a pull element of the at least one Bowden cable; and
at least one clamping plate which has at least one clamping slot and/or at least one clamping groove for clamping a Bowden cable sheath of the at least one Bowden cable.

2. The handpiece as set forth in claim 1, wherein the clamping plate has at least one transverse wall, which has the at least one clamping slot or the at least one clamping groove.

3. The handpiece as set forth in claim 2, wherein the at least one transverse wall has at least two clamping slots or clamping grooves for clamping at least two Bowden cable sheaths.

4. The handpiece as set forth in claim 2, wherein the clamping plate has at least two transverse walls, each of which has at least one clamping slot or at least one clamping groove, wherein the clamping slots or clamping grooves of the at least two transverse walls are arranged one after another in an axial direction of the Bowden cable sheath of the at least one Bowden cable.

5. The handpiece as set forth in claim 4, wherein the at least two transverse walls are interrupted and connected to one another in such a way that they form at least two strips running in the axial direction of the Bowden cable sheath.

6. The handpiece as set forth in claim 1, wherein the at least one clamping slot or the at least one clamping groove tapers from an opening toward a clamping region.

7. The handpiece as set forth in claim 1, wherein the at least one clamping slot or the at least one clamping groove is designed in a stepped manner.

8. The handpiece as set forth in claim 1, wherein the clamping plate has at least two clamping slots or clamping grooves of different widths.

9. The handpiece as set forth in claim 1, wherein the clamping plate has a plurality of transverse walls, each of which has two pairs of clamping slots or clamping grooves with pairs of different widths, wherein, in a first installation position of the clamping plate, the clamping slots or clamping grooves with a first width and, in a second installation position, the clamping slots or clamping grooves with a second width point in a straight line to the control element.

10. The handpiece as set forth in claim 1, wherein the at least one clamping slot or the at least one clamping groove has a constriction formed by at least one latching projection.

11. The handpiece as set forth in claim,
wherein the at least one clamping slot or the at least one clamping groove has a constriction formed by at least one latching projection; and
wherein at least one of the at least two transverse walls has at least one clamping slot or at least one clamping groove which has a constriction formed by at least one latching projection.

12. The handpiece as set forth in claim 1, wherein the handpiece has at least one counter-holder for holding the Bowden cable sheath in the at least one clamping slot or the at least one clamping groove.

13. The handpiece as set forth in claim 12, wherein the at least one counter-holder is designed as a further clamping plate.

14. The handpiece as set forth in claim 1, wherein the clamping plate has at least one latching hook for holding the Bowden cable sheath in the at least one clamping slot or the at least one clamping groove.

15. The handpiece as set forth in claim 1, wherein at least one attachment point of the pull element on the control element is offset from a straight continuation of an axial direction of the at least one Bowden cable sheath in the region of the clamping plate, in such a way that the at least one Bowden cable sheath is preloaded into a clamping region of the at least one clamping slot or of the at least one clamping groove.

16. A flexible endoscope or flexible endoscopic instrument comprising:
a shaft which comprises a shaft portion configured to be angled by means of at least one Bowden cable; and
a handpiece which is arranged at a proximal end of the shaft and has a control element which, in order to actuate the at least one Bowden cable, is connected to a pull element of the at least one Bowden cable, wherein a Bowden cable sheath of the at least one Bowden cable is non-positively connected to the handpiece.

17. The flexible endoscope or flexible endoscopic instrument as set forth in claim 16, wherein the handpiece has at least one clamping element for clamping the Bowden cable sheath of the at least one Bowden cable.

18. The flexible endoscope or flexible endoscopic instrument as set forth in claim 16 wherein the Bowden cable sheath of the at least one Bowden cable is clamped in the at least one clamping slot or the at least one clamping groove.

19. A method for assembling a flexible endoscope or a flexible endoscopic instrument, comprising the steps of:
providing a shaft which comprises a shaft portion which can be angled by means of at least one Bowden cable;
providing a handpiece which has a control element for actuating a pull element of the at least one Bowden cable; and
non-positively connecting a Bowden cable sheath of the at least one Bowden cable to the handpiece.

20. The method as set forth in claim 19, wherein the handpiece includes a control element for actuating a pull element of the at least one Bowden cable; and at least one clamping plate which has at least one clamping slot and/or at least one clamping groove for clamping a Bowden cable sheath of the at least one Bowden cable, and the Bowden cable sheath of the at least one Bowden cable is non-positively connected to the handpiece by being pressed into the at least one clamping slot or the at least one clamping groove.

21. The method as set forth in claim 19, wherein the Bowden cable sheath and/or the clamping plate and/or an additionally introduced plastic material is at least partially heated.
